(19) United States
(12) Patent Application Publication
(10) Pub. No.: US 2024/0216035 A1
(43) Pub. Date: Jul. 4, 2024

(54) Needle Adaptor for Plasma Generating Handpiece

(71) Applicant: Clear Intradermal Technologies, Inc., New York, NY (US)

(72) Inventors: Martin E. SCHMIEG, Delray Beach, FL (US); Craig LOFSTUEN, Waconia, MN (US); Kimberly LINTGEN, Maple Grove, MN (US); William R. BETTEN, Woodbury, MN (US); James W. WINKELMAN, Chestnut Hill, MA (US)

(73) Assignee: Clear Intradermal Technologies, Inc., New York, NY (US)

(21) Appl. No.: 18/562,720

(22) PCT Filed: May 19, 2022
(86) PCT No.: PCT/US2022/030036

§ 371 Date: Nov. 20, 2023

Related U.S. Application Data
(60) Provisional application No. 63/191,115, filed on May 20, 2021.

(51) Int. Cl. A61B 18/04; A61B 18/00; A61B 18/14

(52) U.S. Cl. CPC A61B 18/042; A61B 18/1477; A61B 2018/00172; A61B 2018/0047; A61B 2218/007

(57) ABSTRACT

Methods and devices are disclosed for adapting plasma generating systems for used with one or more needles, such that a plasma can be generated at the distal end or tip of the needle(s). An adaptor with an energy conductor is employed to deliver energy to the tip of the needle at a target site. A fluid, e.g., a liquid, gas or liquid/gas mixture, can also be delivered by the adaptor to the needle to provide an ionizable substance for plasma formation and/or a mobilization fluid to facilitate removal of material that has been plasma-treated. The adaptors can be used, for example, in conjunction with plasma generating handpiece to degrade and/or dislodge tattoo in particles in a tattooed region of a subject's dermis.

### CROSS-REFERENCE TO RELATED APPLICATIONS

This application is a U.S. national stage application of PCT/US2022/030036, filed May 19, 2022, which claims priority to U.S. Provisional Pat. App. No. 63/191,115, filed May 20, 2021, which are herein incorporated by reference in their entireties.

### BACKGROUND

It has recently been discovered that cold plasma can be used to degrade and/or dislodge tattoo ink particles embedded in the dermis during tattooing. See, commonly-owned U.S. Pat. No. 10,716,611 entitled “SYSTEMS AND METHODS FOR TATTOO REMOVAL USING COLD PLASMA;” and U.S. patent application Ser. No. 16/711,545 entitled “SYSTEMS AND METHODS FOR TATTOO REMOVAL USING AN APPLIED ELECTRIC FIELD;” Ser. No. 16/711,549 entitled “SYSTEMS AND METHODS FOR TATTOO REMOVAL USING AN ELECTRO-KINETIC APPLICATOR;” Ser. No. 16/902,767 entitled “SYSTEMS AND METHODS FOR TATTOO REMOVAL USING COLD PLASMA;” Ser. No. 16/938,694 entitled “TATTOO REMOVAL USING A LIQUID-GAS MIXTURE WITH PLASMA GAS BUBBLES;” and Ser. No. 17/527,353 entitled “TATTOO REMOVAL USING IRRADIATION AND FLUID EXTRACTION,” the disclosures of all of the above are incorporated herein in their entirety by reference.

Various cold plasma generating systems are known, including several systems that employ handheld applicators. For example, the Piezobrush PZ2™ and PZ3™ handpieces manufactured by Relyon Plasma GmbH of Regensburg, Germany, can deliver cold plasma to surfaces for various industrial purposes, such as disinfecting, cleaning, and functionalizing a range of surfaces, e.g., to prepare them for bonding, painting and printing. The Relyon systems employ a piezoelectric crystal at their distal end to generate the plasma. However, such plasma generating handpieces are ill-suited for delivery of cold plasma to subsurface regions, such as a subject's tattooed dermis.

Combining a plasma generating handpiece and needle or probe-like structures is difficult because of the need to efficiently transfer energy between a piezoelectric crystal (or other plasma generating element) in the handpiece and the extended tip of a needle or probe in order to initiate a plasma at a subsurface or hard-to-reach site. Direct integration of a needle and handpiece also poses a problem for reuse of the device, since the entire integrated unit would need to be re-sterilized after each use. It would therefore be advantageous to have a needle securing element that could be coupled to a plasma generating handpiece, such that the needles can be disposable and the needle securing element can be re-sterilizable separately from the handpiece or be inexpensive enough to be disposable after use.

Moreover, during the course of plasma treatment to remove a tattoo it can be desirable to employ multiple needles, e.g., needles of different gauges, at various stages of the procedure. Accordingly, an adaptor element that could accommodate different needles and facilitate quick exchange of needles would be advantageous.

### SUMMARY

Methods and devices are disclosed for adapting plasma generating systems for use with one or more needles, such that a plasma can be generated at the distal end or tip of the needle(s). An adaptor with an energy conductor is employed to deliver energy to the tip of the needle at a target site. A fluid, e.g., a liquid, gas or liquid/gas mixture, can also be delivered by the adaptor to the needle to provide an ionizable substance for plasma formation and/or a mobilization fluid to facilitate removal of material that is plasma-treated. The adaptors can be used, for example, in conjunction with a plasma generating handpiece to degrade and/or dislodge tattoo ink particles in a tattooed region of a subject's dermis.

In certain embodiments, needle adaptors for connection to a plasma generating handpiece are disclosed, the adaptor comprising: (1) an adaptor body having a proximal handpiece coupler for coupling to a plasma generating handpiece; (2) a needle connector at a distal end of the adaptor body for connection to one or more needles; (3) an energy conductor disposed at least partially within the adaptor body and extending longitudinally between the plasma generating handpiece and the needle coupler; and (4) a fluid line connector for connection to a fluid supply line. The energy conductor can further comprise at least one internal fluid passageway such that a fluid can flow through the energy conductor to the needle coupler and the fluid and plasma generating energy can be directed via at least one needle to a target site.

The handpiece coupler of the adaptor body can comprise at least one annular groove, annular lip, threaded connector or luer-lock type connector for coupling to a mating connector on the handpiece. The needle connector of the adaptor further can include a threaded connection that couples to a mating connection on a needle. For example, the needle connector can comprise a male threaded segment of the energy conductor that couples to a female threaded collar that is joined to one or more needles. The adaptor body can further comprise a threaded connection that couples to a mating connector on the fluid supply line. For example, the adaptor body can comprise a female threaded connection that couples to a mating male threaded connector on the fluid supply line.

The energy conductor can have a longitudinal lumen configured to deliver fluid to at least one needle. The energy conductor can also include at least one lateral lumen to receive fluid from a fluid delivery line and deliver the fluid to the longitudinal lumen of the energy conductor. The lateral and longitudinal lumens can thus provide the passageway for delivery of one or fluids to the needle tip(s).

In certain embodiments, the adaptor can also include at least one needle joined to the adaptor. For example, disposable needles can be employed having a collar configured to connect to the needle connector of the adaptor body. The adaptor can be disposable as well as the needle(s). Alternatively, the adaptor can be constructed of materials that can be re-sterilized and reused. Needles of various dimensions can be used during a procedure by unscrewing a first needle and replacing it with a needle of a different size. For example, the needles can vary from 3 gauge to 34 gauge, more preferably from about 14 gauge to about 30 gauge (French scale). In some embodiments, the adaptor can accommodate different needles and facilitate quick exchange of needles during a treatment procedure.

In one exemplary embodiment, a needle adaptor for connection to a plasma generating handpiece can comprise: (1) an adaptor body having a proximal handpiece coupler for coupling to a plasma generating handpiece and a threaded connection that couples to a mating connector on a fluid supply line; (2) a needle connector at a distal end of the adaptor body for connection to one or more needles, wherein the needle connector further comprises a threaded connection that couples to a mating connection on a needle; (3) an energy conductor disposed at least partially within the adaptor body and extending longitudinally between the plasma generating handpiece and the needle coupler; and (4) a fluid line connector for connection to a fluid supply line, wherein the energy conductor further comprises at least one internal fluid passageway such that a fluid can flow through the energy conductor to the needle coupler and the fluid and plasma generating energy can be directed via at least one needle to a target site.

In another aspect of the disclosure, methods of delivering plasma and at least one fluid to a target site can comprise the steps of (1) connecting a needle adaptor to plasma generating handpiece, the needle adaptor comprising an adaptor body having a proximal handpiece coupler for coupling to a plasma generating handpiece, a needle connector at a distal end of the adaptor body for connection to at least one needle, an energy conductor disposed at least partially within the adaptor body and extending longitudinally between the plasma generating handpiece and the needle coupler; and a fluid line connector for connection to a fluid supply line; (2) connecting a fluid supply line to the fluid line connector of the adaptor; (3) disposing a distal end of said at least one needle at a target site; (4) delivering fluid from the fluid supply line to a target site via an internal fluid passageway in the energy conductor; and (5) activating the plasma generating handpiece to deliver energy to the at least one needle connected to the distal end of the energy conductor, whereby a plasma is formed at the distal end of the needle.

In certain embodiments, the fluid can comprise one or more gases, such as at least one gas selected from air, carbon dioxide, oxygen, nitrogen, helium, argon, neon, xenon, and krypton. In other embodiments, the fluid can comprise a liquid, such as at least one liquid selected from water, saline, and buffered aqueous solutions. In yet other embodiments, the fluid comprises a liquid-gas mixture. In yet other embodiments more than one fluid can be delivered to a target site. For example, a mobilization fluid can be delivered before, during or after a fluid that provides the ionizable material for plasma ignition.

The plasma generated by the disclosed adaptors is preferably a cold atmospheric plasma. In certain embodiments, the target site is a tattooed region of a subject's dermis. The method can further comprise a step of pretreating the target region by injecting the region with a mobilization fluid via the needle adaptor before plasma generation.

The methods can further comprise a step of applying suction to the target region to extract degraded or dislodged tattoo ink particles following plasma generation. Extraction of plasma treated material, e.g., tattoo ink or degradation products thereof, can be achieved by reversing the fluid flow in the fluid supply line (applying negative pressure via the fluid supply line and adaptor) or a separate fluid extraction device can be utilized.

In some embodiments, methods and systems are disclosed for removing a tattoo from a subject's skin by application of a cold plasma that is delivered via the adaptor and needle. For example, a gas, from which the plasma will be generated, can be delivered to a target site via a fluid supply line that connects to a passageway within the adaptor in fluid communication with the lumen or one or more hollow needles. This process is sometimes referred herein to as “gas-only cold atmospheric plasma (CAP)”. For further details on CAP treatment, see commonly owned U.S. Pat. No. 10,716,611, herein incorporated by reference in its entirety.

Alternatively, a liquid gas mixture can be delivered by the system in a process sometimes referred herein to as fluid cold atmospheric plasma (F-CAP). For example, a tattoo can be removed from a subject's skin by delivering a liquid-gas mixture to a target tattoo site within a dermal region of the skin and then activating the liquid-gas mixture at the needle tip whereby tattoo ink particles are degraded or dislodged by plasma gas bubbles in the liquid at the target tattoo region. The step of forming an activated liquid-gas mixture can further comprise activating a liquid with entrained gas bubbles by applying a high energy electric field to the liquid to induce plasma formation in the gas bubbles.

In certain embodiments, the plasma comprises a cold atmospheric plasma, wherein the plasma applies energy to the target tattoo region without raising the temperature of the target region more than 4 degrees C. The plasma can be formed from at least one gas selected from air, carbon oxide, oxygen, nitrogen, helium, argon, neon, xenon, and krypton. When a liquid/gas mixture is employed, the liquid component of the activated liquid-gas mixture can comprise at least one liquid selected from water, saline, and buffered aqueous solutions. In certain embodiments, the liquid-gas mixture can comprise water with dissolved carbon dioxide. The liquid component can also comprise one or more surfactants, local anesthetics, anti-infective agents, antiseptic agents, anti-inflammatory agents, or combinations thereof.

The activated liquid-gas mixture, or fluid cold atmospheric plasma (F-CAP) can also be applied in conjunction with the gas-only cold atmospheric plasma (CAP). For example, the target tattoo region can be treated first by a CAP treatment, then by F-CAP, or vice-versa. Between CAP and F-CAP treatment, one or more additional mobilization or extraction steps can be practiced.

In addition to conveying the plasma gas bubbles to the target tattoo region of the dermis, the liquid component of the activated liquid-gas mixture can serve as a mobilization fluid such that treatment of tattoo-containing cellular structures and mobilization of dislodged tattoo ink particles occurs concurrently. Alternatively, the methods of the present disclosure can include the step of pretreating the target tattoo region by injecting the region with a separate mobilization fluid. For example, a fluid such as distilled water or saline can be delivered (e.g., injected) to the target tattoo region prior to plasma treatment. A series of blebs can be formed in the skin by such injections, which can assist in registration of the treatment apparatus and/or permit outgassing of gas during or following treatment. The pretreatment mobilization fluid that remains in the dermis can also facilitate extraction of dislodged or degraded ink particles following treatment.

The step of delivering the activated liquid-gas mixture to a target tattoo region can further comprise accessing the target tattoo region via at least one hollow needle inserted into the subject's skin. In some embodiments, multiple needles can be used to deliver the active liquid gas mixture. The liquid-gas mixture can be formed prior to plasma generation, during plasma generation or following plasma generation. For example, a concentric dual lumen tubular plasma generator can include separate passageways for the liquid (e.g., in an inner lumen) and the gas that will be ignited into a plasma (e.g., in an outer lumen). The liquid and plasma can be mixed in the plasma generator, or at the entry into a delivery needle or other treatment applicator or even in the dermis.

The method can further comprise applying suction to the target region, either via a separate device or via the treatment component. For example, dual lumen needles can again be employed with one lumen utilized to deliver the activated liquid-gas mixture and another lumen utilized to extract dislodged or degraded ink particles via suction. Extraction of the ink particles can be achieved via suction of a mobilization fluid and/or suction of a natural bodily fluid containing the particles from the target region.

The step of forming a plasma can further comprise igniting a plasma in a gas by a high strength electric field so that gas atoms are stripped of at least some of their orbital electrons. In certain embodiments, the plasma can be initiated by applying an alternating electric field having at least one frequency ranging from about 1 kHz to 100 MHz. The plasma initiation step can further comprise delivering between about 0.1 and 10 microamperes, or between about 1 and 10 microamperes, of alternating current. The plasma initiation step can also comprise applying a DC voltage between about 1 and 10 kV, or between about 4 and 6 kV.

The methods disclosed herein can further comprise delivering a separate mobilization fluid to the target region before, during or after delivering the plasma to a target tattoo region. This mobilization fluid can comprise at least one of sterile water, a saline solution, or a buffered aqueous solution and optionally one or more surfactants, local anesthetics, anti-infective agents, antiseptic agents, anti-inflammatory agents, or combinations thereof.

In another aspect of the disclosure, systems for removing a tattoo from a subject's skin are disclosed, the system comprising: (1) a plasma generating handpiece; and (2) an adaptor as disclosed herein with one or more fluid conduits.

The system can further comprise a mobilization fluid delivery component for delivering a liquid either from the liquid source or from an alternative liquid source to the target tattoo region either before, during or after delivery of the activated liquid-gas mixture. In one embodiment, the system can further comprise a syringe and needle to deliver of the mobilization fluid.

The system can also comprise an extraction component. In certain embodiments, the extraction component applies suction to the subject's tattooed dermis during or after the application of the activated liquid-gas mixture with plasma gas bubbles.

The system can also include one or more needle applicators configured to connect to the adaptor and to pierce skin and deliver fluid(s) to a tattoo region in a subject's dermis. For example, the treatment applicator can be a hollow needle with a tip, from which the fluid is applied to the target tattoo region. The hollow needle can be a single lumen needle or a multi-lumen, e.g., a multiple sheathed, needle. The treatment system can also include an array of needles, each capable of delivering the activated liquid-gas mixture to the target tattoo region. The treatment applicator can comprise a removable, replaceable and/or disposable cartridge with one or more needles configured to penetrate the subject's tattooed skin.

In certain embodiments, the treatment applicator can further comprise a kinetic actuator that induces movement of the treatment applicator during treatment. For example, the kinetic actuator can cause an active tip of the needle(s) to penetrate and at least partially withdraw from the target region or to laterally vibrate within the target region, e.g., at a rate from about 0.01 Hz to 10 kHz, more preferably from about 0.1 Hz to about 1 kHz, or at a rate of at least 10 times per minute.

The methods and systems of aspects of the disclosure apply energy in the form of a cold plasma at a strength and duration to chemically degrade tattoo ink particles in some embodiments. The applied energy can also rupture cell membranes of tattoo ink-bearing macrophages in the dermis and/or disrupt the extracellular dermal matrix to release tattoo ink particles entrapped within the cells or extracellular matrix.

The plasma generator can be connected to a power supply, operating under the control of a controller to deliver electrical energy capable of igniting a plasma in at least some of the gas atoms or molecules. The plasma can be formed by applying an alternating electric field having at least one frequency ranging from about 1 kHz to 100 MHz. For example, the active electrode can deliver between about 0.1 and 10 microamperes, optionally between about 1 and 10 microamperes of alternating current and/or a voltage between about 1 and 10 kV, optionally between about 4 and 6 kV.

Alternatively, or in addition, the power supply can supply electrical energy to the plasma generator as a DC voltage. In certain embodiments, the power supply can deliver a pulsed DC current having a pulse repetition rate ranging from about 1 kHz to 100 MHz. For example, an active electrode connected to the power supply can deliver DC pulses at between about 0.1 and 10 microamperes, optionally between about 0.1 and 1 microamperes and/or at a voltage between about 1 and 10 kV, optionally between about 4 and 6 kV.

The plasma can be delivered without raising the temperature of the target region more than 4 degrees C.

The methods of aspects of the disclosure preferably also include the steps of mobilizing and extracting dislodged or degraded ink particles in some embodiments. For example, the step of mobilizing ink particles can further comprise delivering a mobilization fluid to the target region. The mobilization fluid can include at least one of sterile water, a saline solution, or a buffered aqueous solution as well as one or more surfactants. The mobilization fluid can also include local anesthetics, anti-infective agents, antiseptic agents, anti-inflammatory agents, or combinations thereof. The extraction step can include extracting ink particles via suction of a mobilization fluid or a natural bodily fluid containing the particles from the target region.

Systems according to aspects of the disclosure can further include an extraction component. The extraction component can apply suction to the subject's tattooed dermis during and/or subsequent to application of the plasma.

In some embodiments, the plasma generating handpiece, adaptor and a fluid delivery component form a treatment component. In other embodiments, the extraction component is integrated into a single treatment component. The treatment component can utilize one or more hollow needles with tips, from which a fluid is applied to the target tattoo region. The hollow needle can be a multiple sheathed needle and, in certain embodiments, the treatment component can comprise a cartridge unit with one or more needles which penetrate the subject's tattooed skin. The cartridge unit can be removable, replaceable, and/or disposable.

In some embodiments, cold plasma is applied via the treatment component to the tattooed dermis and surrounding tissue under the control of a skilled/trained operator or technician and the treatment is applied with a high level of precision. In certain embodiments, all or a portion of the tattoo ink particles are dislodged or degraded, and extracted from the subject's tattooed dermis, to render the tattoo undetectable, invisible, and/or non-discernible to the naked eye.

Methods and systems using applied plasma to remove tattoos from a subject have been developed based on application of an alternating current (AC) or a pulsed direct current (DC) electric field to form a plasma which can dislodge and degrade tattoo ink particles trapped within a subject's dermis to facilitate the removal of the mobilized ink particles and/or degradation products thereof from the subject's dermis and surrounding tissues and render the tattoo invisible, non-discernible, and/or undetectable.

The plasma can be applied at a fluence and duration sufficient to chemically degrade tattoo ink particles, or at a fluence and duration sufficient to disrupt the extracellular dermal matrix, or at a fluence and duration sufficient to rupture cell membranes of tattoo ink-bearing macrophages and release tattoo ink particles entrapped therein. Preferably, the plasma is applied without raising the temperature of the target region more than 4 degrees C. The plasma can also be applied in conjunction with electrical energy.

In certain embodiments, the method can further include the steps of mobilizing and extracting dislodged or degraded ink particles. For example, degraded ink particles can be mobilized by delivering a mobilization fluid to the target region. The mobilization fluid can include at least one of sterile water, a saline solution, or a buffered aqueous solution, and optionally can further include one or more surfactants, or one or more local anesthetics, anti-infective agents, antiseptic agents, anti-inflammatory agents, or combinations thereof.

The extraction step can include extracting degraded ink particles via suction of a mobilization fluid or a natural bodily fluid containing the particles from the target region. The method can also repeat the mobilizing and extracting steps, or cycle the plasma application, mobilization and extraction steps. The treatment applicator and/or mobilization and extraction elements can also be in motion during operation, e.g., vibrating or oscillating in depth, to further augment their function and/or expose a larger portion of the target region.

In certain embodiments, the system can also employ a plurality of electrodes disposed in an array with the electrodes separated from each other by a distance sufficient to achieve a generally uniform electric field over at least a portion of target region by overlapping fields For example, in various embodiments, the spacing between adjacent electrodes can be less than about 5, or less than about 4 mm, or less than about 3 mm, or less than about 2 mm. In certain embodiments, the spacing can be between 0.25 mm to 2 mm, e.g., about 1 mm for facilitating the interaction of electric fields generated by the electrodes. In some embodiments, the electrode array can include multiple electrodes arranged in rows and/or columns, for example, at least 9 active electrodes, or optionally at least 16 electrodes, or optionally at least 24 electrodes, arranged in a honeycomb pattern. The electrodes can augment the application of F-CAP by providing additional energy to the target tattoo region.

Additionally, the system can further include a mechanical actuator or oscillator connected to the one or more active electrodes to permit movement during operation, e.g., vibratory or oscillatory movement of the electrode during treatment.

In one preferred embodiment, F-CAP is applied to the subject's dermis via one or more needles or probe-like structures that penetrate the subject's tattooed skin. The plasma can be applied so that the energy interacts with constituents present within the dermis such as, but not limited to, the tattoo ink particles themselves, macrophages, fibroblasts, cell membranes, collagen fibers, and capillaries and other cellular and non-cellular constituents of the dermis which have trapped the tattoo ink particles in such a manner as to effectively disrupt the tissue components and dislodge the trapped tattoo ink particles. The plasma gas bubbles may also induce degradation of certain types of the ink particles, which are composed of organic and/or inorganic pigments, dyes, and/or chromophores and give color to the ink particles. In preferred embodiments, the electrical energy both degrades and dislodges the trapped ink particles without causing any damage or any significant amount of thermal or other type of irreparable damage to the exposed dermis or other surrounding tissue.

In some embodiments, the cold plasma effectively dislodges and/or degrades all or a portion of the tattoo ink particles during a single or multiple tattoo removal treatment. Multiple treatments may be applied wherein the number of treatments depends on factors such as the size and complexity of the tattoo and on the health of the individual and/or individual's skin. As noted above F-CAP and CAP can be applied sequentially together.

In some embodiments, the dislodged ink particles and degradation by-products thereof can be mobilized to remove them from the subject's dermis and surrounding tissues prior to their recapture by the natural protection mechanisms of the skin, which can otherwise result in a shadowing effect or prior to their transport through the lymphatic channels and deposition in lymph nodes.

In some embodiments, the mobilization step involves the delivery of a pharmaceutically acceptable mobilization fluid which facilitates the removal of the dislodged and degraded ink particles and by-products thereof. The mobilization fluid delivered to the treated dermis is extracted in a subsequent extraction step such as by the application of suction. The extraction of the mobilization fluid containing the dislodged and degraded ink particles from the dermis and surrounding tissues removes the tattoo from the skin.

All or a portion of the dislodged and degraded tattoo ink particles and by-products thereof can be extracted from the subject's tattooed dermis during an extraction step. By degrading, dislodging and removing the tattoo ink particles, the tattoo on skin treated according to the method described herein becomes undetectable, invisible, and/or non-discernible to the naked eye. In certain other embodiments, the cold plasma can degrade all or a portion of the tattoo ink particles and the degradation by-products are converted into colorless components and the tattoo becomes undetectable, invisible, and/or non-discernible to the naked eye. In such embodiments, treatment of the tattoo ink particles with applied plasma gas bubbles may render the ink particles down to their colorless atomic, molecular, and/or gaseous components, such as carbon dioxide or water. In some embodiments, the colorless components may not need to be removed or otherwise extracted from the skin if the tattoo has otherwise been rendered undetectable, invisible, and/or non-discernible to the naked eye. In other embodiments, the dislodged and degraded ink particles and degradation by-products thereof which are rendered into colorless components may be absorbed by natural processes from the interstitial fluid of the dermis or elsewhere in the body.

The extraction of the degraded and dislodged ink particles and by-products thereof from the subject's skin is advantageous as the ink particles, components and degradation by-products thereof may have toxic properties which can potentially have harmful effects if absorbed by the subject's body.

These and other features of the applicant's teachings are set forth herein.

### BRIEF DESCRIPTION OF THE DRAWINGS

The skilled person in the art will understand that the drawings, described below, are for illustration purposes only. The drawings are not intended to limit the scope of the applicant's teachings in any way.

FIG. 1A is a schematic perspective view of a prior art plasma generating handpiece;

FIG. 1B shows a similar perspective view of a plasma generating handpiece with one embodiment of a needle adaptor attached to the handpiece;

FIG. 2 is a side view of the needle adaptor according to the disclosure;

FIG. 2A is a top view of the adaptor of FIG. 2;

FIG. 2B is a bottom view of the adaptor of FIG. 2;

FIG. 2C is a perspective view of the adaptor of FIG. 2; and

FIG. 3 is a cross-sectional side view of the adaptor of FIG. 2.

It should be understood that a number of modifications can be made to the system and/or components shown in the Figures. For the purposes of clarity, not every component is labeled in every illustration of the system and/or components as shown in the figures, nor is every component of each embodiment shown where illustration is not required to allow one of ordinary skill to understand the system and/or components.

### DETAILED DESCRIPTION

Permanent tattoos are created by piercing the skin with needles or similar instruments to mechanically deliver an ink, which includes small particles of pigments/dyes suspended in a carrier, into the dermal layer of the skin. The creation of a permanent tattoo requires the insertion/implantation of pigments, dyes, and/or chromophores into the dermis which are not dissolvable and/or biodegradable. Following mechanical insertion of the ink particles and during the healing process, the majority of the ink particles that remain in the dermis and that have not otherwise been expelled from the skin or absorbed by the body in the healing process, 70-80%, are engulfed by phagocytic skin cells (such as fibroblasts and macrophages) or retained in the extracellular matrix of the dermis and the remaining ink particles are found such that 10-15% of the ink particles lie flattened on collagen fibers and 5-10% of the ink particles lie attached on the serosal side of capillaries.

Despite the wide acceptance and popularity of permanent tattoos, there is a significant demand for the removal of tattoos. Removal of tattoos, however, represents a complex process that most typically involves the use of lasers designed for aesthetic skin treatments and/or other mechanical removal techniques. The current state-of-the-art for tattoo removal is performed using a variety of lasers which induce degradation and absorption by the body of the inks to achieve tattoo removal. The laser conditions require matching the laser frequencies to the particles according to their size, composition, color, and depth in the dermis. The laser is applied to the tattoo such that the pigments, dyes, and/or chromophores of the ink particles absorb the laser light and the laser pulses dissociate and degrade the pigments, dyes, and/or chromophores components of the ink particles into small(er) fragments. The fragmented ink components may become small enough to be absorbed by the body and removed from the dermis. Nonetheless, laser-based removal of tattoos has several shortcomings. For example, lasers induce heating of the skin and can cause burns as well as other undesirable tissue damage which can cause some scarring or color variations that are likely to remain after healing. Current laser-based procedures for tattoo removal may therefore be somewhat ineffective at complete removal of tattoo inks, require multiple treatments at a high cost, cause pain, and can result in scarring, disfigurement, and depigmentation of the treated skin.

Therefore, it would be advantageous to provide systems and methods for tattoo removal using non-laser-based approaches. It would also be advantageous to provide methods that enable removal/extraction of the degraded ink components, such as dyes, pigments and other chromophores, from the body to reduce absorption by the body of potentially harmful/toxic chemicals. Therefore, the present teachings provide systems and methods for removing a tattoo from a subject by degrading ink particles trapped within the dermis. More specifically, the present teachings provide adaptors and methods for use with conventional cold plasma generating handpieces.

The present teachings further provide systems and methods which allow for the extraction of the residue of treated tattoo ink particles, which may have toxic properties, and of other degradation components from the subject's skin tissues. The adaptors and methods of the present teachings provide methods for removal of tattoos in one or more treatments, which are effectively less painful to the subject being treated than current conventional methods of tattoo removal. Thus, the present teachings provide devices and methods of tattoo removal which can address the limitations of current state-of-the-art removal methods (i.e., laser-based removal systems) to reduce issues with skin scarring, skin color bleaching, and residual tattoo shadowing remaining after removal treatment(s).

Various terms relating to aspects of the present disclosure are used throughout the specification and claims. Such terms are to be given their ordinary meanings in the art, unless otherwise indicated. In order for the present disclosure to be more readily understood, certain terms are first defined below. Additional definitions for the following terms and other terms are set forth throughout the specification.

As used herein, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, unless otherwise clear from context, the term “a” may be understood to mean “at least one.” As used in this application, the term “or” may be understood to mean “and/or.” In this application, the terms “comprising” and “including” may be understood to encompass itemized components or steps whether presented by themselves or together with one or more additional components or steps.

As used herein, the term “bubble” is used to describe the gas component of the activated liquid-gas mixture. The bubbles can be visible or so small as to be invisible, e.g., dissolved gas molecules. The bubbles can take the form of a foam or be part of turbulent liquid-gas flow. However, within the bubbles at least a portion of the gas molecules have been excited to a “plasma” state in which individual atoms or molecules have been stripped of at least some of their orbital electrons.

“Connected,” and “coupled,” as used herein, refers to directly coupling (i.e., connecting) one element (i.e., output) of a system or component to another element (i.e. input) by any suitable means available, such as, for example, through tubing. Optionally, other intervening elements may also be present.

“Color,” as used herein, is broadly defined as a detectable property determined by a substance's electromagnetic absorption and/or emission in the visible spectrum.

“Colorless,” as used herein, refers to when essentially no color can be detected apart from the normal coloration of the surroundings (such as skin or other tissue) by the naked eye under normal lighting conditions, for example, diffuse sunlight or standard artificial lighting.

“Dielectric barrier” discharge as used herein refers to an electrical discharge between electrodes separated by a dielectric material. For example, one or both electrodes can be coated with a dielectric material.

“Dislodged,” as used herein, refers to the release of tattoo ink particles from local skin cells and tissue structures such as cells, membranes, and/or tissues, typically found in the dermis. As used herein, “Dislodge,” “Dislodged,” “Dislodgement,” or other variations also encompass degradation of tattoo ink particles.

“Degrade,” “Degraded,” “Dislodgement,” and the like as used herein, refers to the dislodgement of tattoo particles by the breakdown of the organic and/or inorganic components of tattoo ink particles due to interaction with the applied cold plasma energy via processes that include, but are not limited to, oxidation, reduction, fragmentation, electron decomposition, ion decomposition, or other degradation pathways. Degradation generally refers to a breakdown of a colored organic pigment, dye, or chromophore and/or to the breakdown of the particle size of colored inorganic ink particles which causes them to become colorless. Degradation can come about through the disruption of crystals or amorphic masses of elements such carbon, or by the breaking of chemical bonds in organic or inorganic compounds.

“Pigment, dye, or chromophore,” as used herein, are terms that refer to organic and/or inorganic substance(s) which are colored and impart color to a tattoo ink. The color may result from substances which contain heavy metals such as mercury (red), lead (yellow, green, white), cadmium (red, orange, yellow), Chromium (green), cobalt (blue), aluminum (green, violet), titanium (white), copper (blue, green), iron (brown, red, black), barium (white), substances which contain metal oxides such as ferrocyanide and ferricyanide (yellow, red, green, blue), substances such as organic chemicals/compounds such as azo-containing chemicals (orange, brown, yellow, green, violet), naptha-derived chemicals (red), substances such as carbon (i.e., soot or ash) for black ink, and other color compounds which may contain antimony, arsenic, beryllium, calcium, lithium, selenium and sulfur. The pigments, dyes, or chromophores of a tattoo ink are typically dispersed or suspended in a carrier medium which together are delivered to the dermis. The most typical carrier constituents are ethyl alcohol and water, but may be denatured alcohols, methanol, rubbing alcohol, propylene glycol, and/or glycerin.

“Plasma,” as used herein connotes a state of matter in which one or more atoms or molecules have been subjected to sufficient energy to strip at least some of their orbital electrons. The transition from gas to plasma is also referred to as ionization.

“Invisible,” as used herein, refers to the state of tattoo inks that show essentially no color which can be detected (such as in a tissue) apart from the normal coloration of the surroundings (such as skin or other tissue) by the naked eye under normal lighting conditions, for example, diffuse sunlight or standard artificial lighting.

“Non-discernible and undetectable,” are used interchangeably and refer to a substance (i.e., tattoo ink) rendered invisible to the naked eye under normal lighting conditions, and also invisible to the naked eye, or a device, under any other lighting conditions.

“Removal” of a tattoo as used herein refers to any reduction of the visible appearance of a tattoo. Removal can mean rendering a tattoo non-discernible and undetectable or simply rendering a tattoo less noticeable in its appearance.

“Substantially” refers to a qualitative condition of exhibiting total or near-total extent or degree of a characteristic or property of interest. One of ordinary skill in the art will understand that electrical properties rarely, if ever, go to completion and/or proceed to completeness or achieve or avoid an absolute result. Substantially is therefore used herein to capture a potential lack of completeness inherent therein. Values may differ in a range of values within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than). For example, values may differ by 5%.

“Tattoo,” as used herein, refers to a portion of skin, typically the dermis, which has tattoo ink particles embedded or trapped within.

“Uniform” refers to a qualitative condition of exhibiting similarity in a characteristic or property of interest. “Uniform” is therefore used herein to capture a degree of substantial similarity. Values may differ in a range of values within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than). For example, values may differ by 5%.

The apparatus and methods for tattoo removal described herein are based on application of a DC pulsed or high frequency AC at a sufficient strength and duration to form a plasma capable of dislodging or degrading tattoo ink particles trapped within a subject's dermis and extraction of the mobilized particles and/or degradation products from the subject's dermis. The method includes the steps of: (i) dislodging and/or degrading tattoo ink particles by applying electrical energy to a subject's tattooed dermis; (ii) mobilizing the dislodged and/or degraded ink particles and by-products thereof; and (iii) extracting the dislodged and/or degraded ink particles and by-products thereof from the subject's dermis to render the tattoo undetectable, invisible, and/or non-discernible.

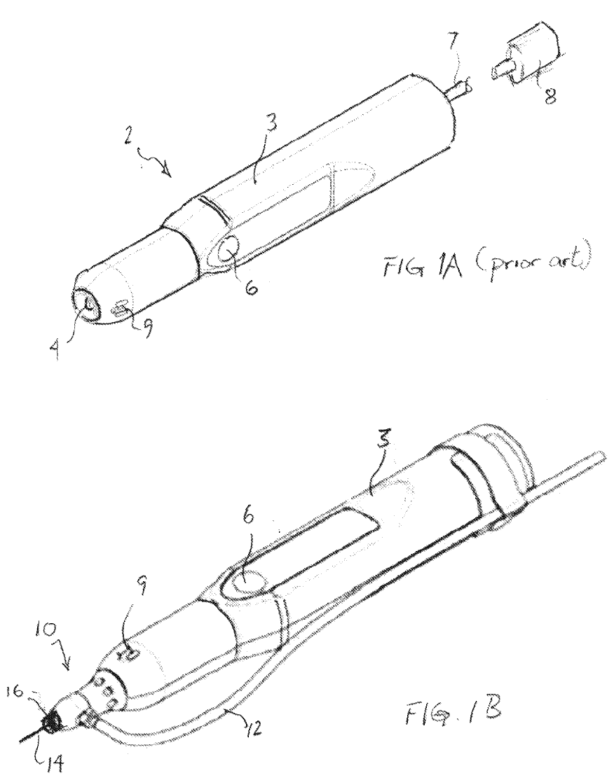

FIG. 1A is a schematic perspective view of a prior art plasma generating handpiece 2. One example of a plasma generating handpiece is the Piezobrush PZ2™ handpiece manufactured by Relyon Plasma GmbH of Regensburg, Germany. Within the handpiece body 3, a piezoelectric crystal 4 is disposed such that it can be activated to initiate a cold plasma discharge. The handpiece can also include controls 6 disposed on the surface of the body 3. The handpiece 2 typically further includes a power supply line 7 connectable to a power supply 8.

The inner construction of the handpiece 2 and the electronics to power the piezoelectrical crystal 4 are well known and can be found, for example, in the whitepapers and technical bulletins of Relyon Plasma GmbH. See for example, the PiezoBrush PZ2™ manual available at: https://www.relyon˜plasma.com/wpcontent/uploads/2018/10/F0351301_BA_pi ezobrush_PZ2_ML.pdf, herein incorporated in its entirety by reference. It should be appreciated that other plasma generating handpieces can also be employed with the adaptors disclosed herein. Another example of a prior art plasma generating handpiece is the Piezobrush PZ3™, also manufactured by Relyon Plasma GmbH. A description of the Piezobrush PZ3™ system and its operation can be found, for example, at https://www.relyon-plasma.com/wp-content/uploads/2020/11/201024_whitepaper_piezobrush_PZ3_1.pdf, also herein incorporated in its entirety by reference.

FIG. 1B shows a similar perspective view of a plasma generating handpiece with one embodiment of a needle adaptor 10 attached to the handpiece body 3. The adaptor can be joined to the Piezobrush PZ2™ handpiece utilizing an annular groove on the adaptor (described below) and the handpiece's existing adaptor release clip 9. Various other mechanisms can also be used to connect the needle adaptors disclosed herein to plasma generating handpieces, e.g., an annular lip, threaded connector or luer-lock type connector for coupling to a mating connector on the handpiece body 3. The adaptor 10 is also adapted to receive a fluid supply line 12 (also described below). The adaptor 10 is configured to receive a needle 14 at its distal end. In the illustrated embodiment, the needle 14 is joined to an internally threaded collar 16 that mates with external threads on the distal ends of the adaptor 10 (described further below).

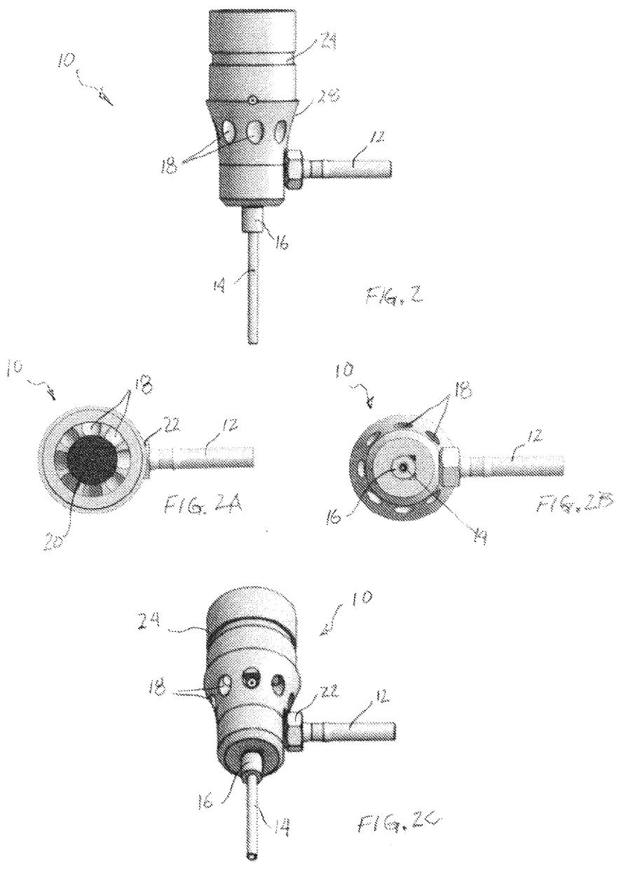

FIG. 2 is a side view of a needle adaptor 10. FIG. 2A is a top view of the adaptor of FIG. 2. FIG. 2B is a bottom view of the adaptor of FIG. 2. FIG. 2C is a perspective view of the adaptor of FIG. 2. The external features of the needle adaptor 10 will be described with reference to these figures. The adaptor 10 can include one or more air inlet ports 18, an energy conductor 20, a fluid line connector 22, a proximal adaptor-to-handpiece coupler 24 and a distal coupler adapted to receive a needle 14.

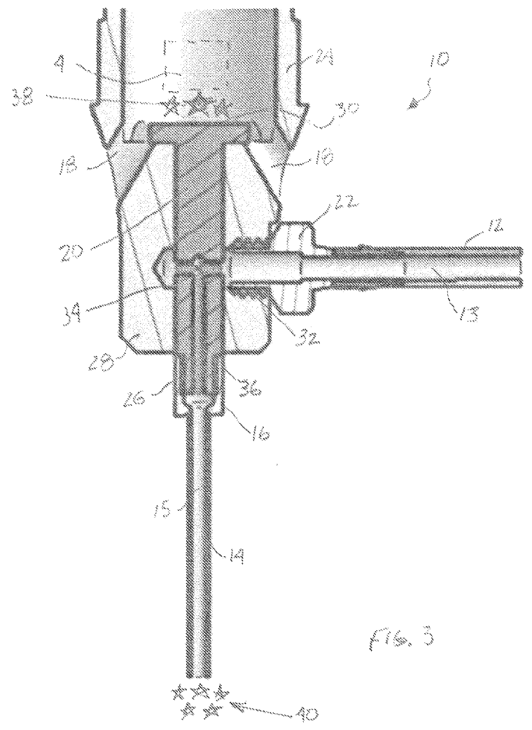

FIG. 3 is a cross-sectional side view of the adaptor of FIG. 2, which further illustrates certain components of the adaptor not shown in FIGS. 2 or FIGS. 2A-2C. As shown in FIG. 3, the energy conductor 20 of adaptor 10 has a shaft portion and an enlarged head that is disposed within the handpiece (not shown) in close proximity to the piezoelectric crystal 4 (shown in dotted lines). In the illustrated embodiment of the adaptor 10, the fluid line coupler 32 is a female-threaded receptacle drilled into the adaptor body 28 that mates with a male-threaded stem of the fluid line connector 22. The fluid line coupler 32 is in fluid communication with a lateral lumen 34 and a longitudinal lumen 36 in the energy conductor 20 that together provide a fluid passageway from the fluid supply line to the lumen 15 of needle 14. In the illustrated embodiment, a female-threaded collar is joined to the proximal end of the needle 14 in order to connect the needle 14 to a male-threaded distal end 26 of the energy conductor 20.

In operation, the piezoelectric crystal is activated and an initial plasma 38 is formed in the space between the piezoelectric crystal 4 and the head 30 of the conductor 20. This initial plasma 38 can be formed by atmospheric gases entering the adaptor via the air inlet ports 18. The enlarged head 30 is designed to capture most of the energy from this initial plasma as electrical energy that is then conducted by the energy conductor 20, collar 16 and needle 14 to the distal tip of the needle where a working plasma 40 is formed at the target site, e.g., at a tattooed region of a subject's dermis.

In certain embodiments, one or more needles are used to pierce the epidermis and deliver plasma to a target tattoo region within the dermis. In this region, tattoo ink particles are present (typically entrapped in cellular structures). The needle delivers a gas or a liquid-gas mixture to the target region such that plasma can act upon the cellular structures that are holding the ink particles and dislodge and/or degrade them.

The gas or liquid/gas mixture is delivered to the tattooed dermis of a subject and induces dislodgement of ink particles trapped by the cells, membranes, and/or other tissue structures of the dermis which are holding the ink particles in place. In preferred embodiments, the plasma is delivered to the dermis via one or more needle or probe-like structures that can penetrate the subject's tattooed skin. Those skilled in the art will be able to determine the penetration depth of the one of more needle or probe-like structures to deliver the plasma to the tattooed dermis.

It is believed that the plasma delivered to a subject's tattooed dermis interacts with constituents present within the dermis such as, but not limited to, macrophages, fibroblasts, other cells, collagen fibers, and capillaries which have trapped the tattoo ink particle, in a sufficient amount to effectively disrupt the local dermal skin cells and tissue structures holding the particles and dislodge the trapped tattoo ink particles from the dermis and surrounding tissues. The plasma also may induce degradation of the ink particles, which are composed of organic and/or inorganic pigments, dyes, and/or chromophores and give color to the ink particles. Such degradation can result from the interaction of the plasma with the organic and/or inorganic components of the ink particles to degrade them via such processes as oxidation, reduction, fragmentation, electron decomposition, ion decomposition, or other degradation pathways.

In preferred embodiments, the plasma dislodges the trapped ink particles without causing a significant amount of thermal or other type of irreparable damage to the subject's dermis or surrounding tissue.

In some embodiments of the method, the exposure time of the dermis to the plasma needed to dislodge and degrade the tattoo ink particles can be as short as one microsecond, but is more preferably a longer period of time, in the range from about one microsecond up to about one hour. In some embodiments, the plasma effectively degrades and dislodges the ink particles at the point of exposure within a period of time of 60 minutes or less, more preferably 10 minutes or less. In certain embodiments, the plasma may effectively dislodge and degrade all or a portion of the tattoo ink particles within a single tattoo removal treatment. In other embodiments, multiple treatments using plasma according to the methods described may be applied. The number of treatments depends on factors such as the area/size and complexity of the tattoo (for example, multi-colored and/or multi-layered tattoo and the age and settling of tattoo inks into lower portion of dermis over time) and on the health of the individual and/or individual's skin. In some non-limiting embodiments, tattooed skin having an area of up to 5 square inches may be treated in as little as one treatment. For tattoos having a larger surface area/size and/or complexity, repeated treatments may be applied with an intervening period time passing between treatments, such as up to one week, up to two weeks, up to three weeks, up to one month, up to two months, or up to three months; longer periods of time may pass between treatments as needed. In preferred embodiments of the method, the temperature of the dermis or other surrounding tissues is not increased by exposure to the plasma.

In certain other embodiments, the temperature of the dermis or other surrounding tissues when exposed to a treatment is not increased at all or significantly, only increasing by about 1° to about 5° C. above normal body temperature, which is below the temperatures needed to induce any significant amount of thermal damage or pain. The application of plasma to the dermis for tattoo removal is not expected to produce blanching and/or bleaching of the subject's natural skin color or pigmentation.

The dislodged ink particles and/or degradation by-products can be mobilized in a mobilization step to remove them from the subject's dermis and surrounding tissues prior to their recapture by the natural protection mechanisms of the skin, which can result in a re-tattooing effect. In some embodiments, the mobilization step involves the delivery of a pharmaceutically acceptable mobilization fluid, preferably through the same adaptor and needle(s) used to deliver the plasma. The mobilization fluid facilitates the removal of the dislodged and degraded ink particles and by-products thereof from the dermis. The mobilization fluid delivered to the plasma treated dermis is extracted in a subsequent extraction step which can be accomplished by any suitable means, such as by the application of suction. Suction, as used herein, refers to at least a partial vacuum created at the ends of the one or more needle or probe-like structures described above, such that the mobilization fluid containing the dislodged and degraded ink particles is drawn away and extracted from the dermis and surrounding tissues. In some embodiments, suction is applied as a continuous suction or, alternatively, suction can be applied as a non-continuous pulsing suction. In some embodiments, no mobilization fluid is administered during or after the treatment and the dislodged ink particles and degradation by-products thereof are removed by extraction (i.e., suction) of natural bodily fluid(s) containing the particles and by-products from the dermis and/or surrounding tissue during the extraction step.

In preferred embodiments, all or a portion of the dislodged and/or degraded tattoo ink particles are extracted from a tattoo during the extraction step. By removing dislodged and degraded tattoo ink particles, the tattoo on skin treated according to the method described becomes undetectable, invisible, and/or non-discernible. By definition, an effective amount of plasma is applied to cause the colors in the original tattoo in the treated area to become undetectable, invisible and/or non-discernible. In some embodiments, treatment of the tattoo ink particles with plasma may render the ink particles down to their colorless atomic, molecular, and/or gaseous components, such as carbon dioxide or water, and the colorless components may not require removal or extraction from the skin if the tattoo has otherwise been rendered undetectable, invisible, and/or non-discernible to the naked eye. In such embodiments, the portion of dislodged and degraded ink particles and degradation by-products thereof which are rendered into colorless components and which remain in the dermis may be absorbed through the interstitial fluid of the body. In such embodiments the method involves dislodging and degrading tattoo ink particles by applying plasma to a subject's tattooed dermis; wherein the energy is applied in an effective amount to a subject's dermis to render the tattoo undetectable, invisible, and/or non-discernible.

The application of the steps of mobilizing the dislodged and/or degraded ink particles and by-products thereof and extracting the dislodged and/or degraded ink particles and by-products thereof from the subject's dermis as described above are optional and determined at the discretion of the skilled technician or operator applying the tattoo removal method to the subject's tattooed skin. Depending on the extent to which the tattoo has been rendered undetectable, invisible, and/or non-discernible by plasma treatment alone the operator/technician may apply the step described above repeatedly in order to further render the tattoo undetectable, invisible, and/or non-discernible.

In some embodiments, the extraction of the degraded and/or dislodged ink particles and by-products thereof from the subject's skin is highly desirable as these may have toxic properties. In contrast to laser-based tattoo removal techniques wherein inks and degradation by-products thereof may remain in situ and/or become absorbed by the subject's body, the methods described herein can result in extraction of these foreign inks and components in order to prevent their absorption by the subject and any potentially harmful effects on health.

In some embodiments of the method, the steps of dislodgement, mobilization, and extraction are performed in sequence. In such embodiments the steps can be performed so as to provide at least one complete cycle which includes the dislodgement, mobilization, and extraction steps. The complete cycle may be repeated any number of times as necessary to effectively remove the tattoo by dislodging and degrading tattoo ink particles from the subject's dermis and tissue. The preferred number of cycles which may be applied are typically in the range of one to 100 cycles, or more.

In certain other embodiments, all of the steps can be applied concurrently. In a non-limiting example, the dislodgement (application of plasma to tattooed dermis), mobilization, which may include the introduction of a mobilization fluid to the dermis, and the extraction step, which involves removal of the mobilization fluid containing the dislodged and degraded ink particles and degradation by-products thereof, or in some instances where no mobilization fluid is used, removes the dislodged and degraded ink particles and degradation by-products thereof directly.

In some other embodiments, the steps of dislodgement and mobilization occur concurrently and are followed by the extraction step and form a cycle which is performed at least one or more times, as necessary to remove the tattoo ink from the subject's dermis and rendering the tattoo undetectable, invisible, and/or non-discernible.

In certain embodiments, the method described above can further include a pretreatment of the surface of the tattooed skin with a mobilization fluid, e.g., water saline or the like. In certain embodiments, an antibiotic solution can be pre-applied in order to prevent the introduction of infectious organisms present on the surface to the skin into the dermis during treatment. In other embodiments, the pretreatment may also include application of topical anesthetics to the surface of the skin in order to prevent or alleviate any potential discomfort during the treatment.

In some embodiments, electrical energy can be applied in conjunction with “cold plasma” that, as used herein refers to a non-thermal or atmospheric plasma, generated by subjecting a gas(es) to a strong electrical field with a rapidly changing polarity to create a plasma which may contain electrons, highly energetic positively or negatively charged ions, and chemically active species such as ozone, hydroxyl radicals, nitrous oxides and other excited atoms or molecules. In particular, cold or non-thermal plasmas are created at or near standard atmospheric pressure and have temperatures which are close to or near room temperature which are non-damaging when applied to tissue. Contacting tissue with a cold plasma does not increase the tissue temperature at all or significantly, typically only by a few degrees or less.

Methods for generating cold plasma as described herein are well-understood by those skilled in the art. Exemplary methods to produce atmospheric cold plasmas include, but are not limited to, arc discharge, corona discharge, dielectric barrier discharge (DBD), capacitive discharge, and piezoelectric direct discharge. Typically, such plasmas are generated from a gas or a mixture of gases which include, but are not limited to, air, oxygen, nitrogen, helium, argon, neon, xenon, and krypton. In certain embodiments, the cold plasma is generated from a mixture of argon and oxygen or a mixture of helium and oxygen. Conditions such as the power, flow rate of gas(es), and the ratio of gases in mixtures used to generate a cold plasma can be optimized as needed to achieve the desired properties of the cold plasma, such as to ensure it is at or near room temperature.

In certain embodiments the power used to generate the plasma is in the range of about 0.1 W to about 150 W, e.g., in a range of about 1 W to 50 W, or in range of about 10 W to about 20 W. In other embodiments, the power used to generate the plasma is in the range of 80 W to about 150 W. In some preferred embodiments, the gas flow rates are in the range of about 0.00001 to about 15 L min-1. The relative percentages of the one or more gases present in the mixture can be any suitable relative percentage necessary to achieve a cold plasma. In preferred embodiments, wherein the plasma generating mixture of gases is composed of oxygen mixed with argon or helium, the percentage of oxygen in the mixture is preferably in the range of about 0.1% to about 5%.

The cold plasma stream generated according to the methods described herein may be delivered and output into the dermis via one or more needle or probe-like structures as a continuous cold plasma jet stream or can be delivered as a discontinuous pulsed cold plasma jet stream. It should be apparent that the details described herein are non-limiting and that other suitable conditions and parameters can be selected and utilized in order to generate and deliver the cold plasma to the dermis.

In certain embodiments, non-limiting examples of the mobilization fluid include sterile water, saline solution, or buffered aqueous solutions. One skilled in the art can readily determine a suitable saline and buffer content and pH for a mobilization fluid/solution to be administered to the dermis of a subject. Representative examples include phosphate buffered saline (“PBS”), Ringer's solution, and sterile physiological saline (0.15 M NaCl).

In certain embodiments, the mobilization fluid can further include surfactants which improve the mobility and removal efficiency of the degraded ink particles and/or degradation by-products thereof. Preferred surfactants include those approved by the U.S. Food and Drug Administration (“FDA”) as GRAS (“generally regarded as safe”) excipients for injection. In certain other embodiments, the mobilization fluid can also include suitable local anesthetics, anti-infective agents, antiseptic agents, anti-inflammatory agents, and combinations thereof.

Surfactants which can be included in the mobilization fluid may be anionic, cationic, amphoteric, and non-ionic surfactants which are pharmaceutically acceptable for use in a human subject. Anionic surfactants include di-(2 ethylhexyl)sodium sulfosuccinate; non-ionic surfactants include the fatty acids such as butyric acid, valeric acid, caproic acid, enanthic acid, caprylic acid, pelargonic acid, caprylic acid, undecylic acid, lauric acid, tridecylic acid, myristic acid, pentadecylic acid, palmitic acid, heptadecylic acid, stearic acid, nonadecanoic acid, arachic acid, isocrotonic acid, undecylenic acid, oleic acid, elaidic acid, sorbic acid, linoleic acid, linolenic acid, arachidonic acid and esters thereof, surfactants in the amphoteric group include substances classified as simple, conjugated and derived proteins such as the albumins, gelatins, and glycoproteins, and substances contained within the phospholipid classification. Amine salts and quaternary ammonium salts within the cationic group also comprise useful surfactants. Synthetic polymers may also be used as surfactants and include compositions such as polyethylene glycol and polypropylene glycol. Hydrophobic surfactants can be used to improve the removal of hydrophobic ink particles and degradation by-products thereof. Hydrophilic surfactants can be used to improve the removal of hydrophilic ink particles and components and degradation by-products thereof. Amphiphilic surfactants can be used to improve the removal of amphiphilic ink particles and components and degradation by-products thereof.

In some embodiments, anesthetic agents can be included in the mobilization fluid such as local anesthetics, such as but not limited to anesthetics such as bupivacaine, ropivacaine, dibucaine, procaine, chloroprocaine, prilocaine, mepivacaine, etidocaine, tetracaine, lidocaine, and xylocaine, and mixtures thereof which can be used alone or in combination with other analgesics.

In some embodiments, antiseptic agents can be included in the mobilization fluid. Exemplary antiseptic agents can be composed of any anti-infective compound that prevents the growth of and/or kills infectious organisms. Antiseptic agents are preferably non-irritating and hypoallergenic, such that they do not cause any adverse reactions to the dermis and surrounding tissue of the subject.

“Anti-infective agent,” as used herein, refers to common antibacterial, antifungal, and antiviral agents which can be include a chemical substance or group of chemical substances that inhibit the growth of, or destroy microorganisms, fungi, and viruses and are used chiefly in the treatment of infectious diseases. In some preferred embodiments, antibiotics can be included in the mobilization fluid. These may help to prevent infection in the dermis and surrounding tissues of the site of tattoo removal. Exemplary antibiotics include, but are not limited to, chloramphenicol, chlortetracycline, clindamycin, erythromycin, gramicidin, gentamicin, metronidazole, mupiroicin, neomycin, polymyxin B, bacitracin, doxycycline, ampicillin, penicillin, silver sulfadiazine, tetracycline, erythromycin, or combinations thereof.

In some embodiments, anti-inflammatory agents can be included in the mobilization fluid. Anti-inflammatory agents can provide beneficial effects during tissue healing and repair. Anti-inflammatory agents can include, but are not limited to, steroidal anti-inflammatory agents such as dexamethasone, budesonide, beclomethasone, and hydrocortisone and non-steroidal Anti-Inflammatory Agents (NSAIDS). NSAIDS typically inhibit the body's ability to synthesize prostaglandins. Prostaglandins are a family of hormone-like chemicals, some of which are made in response to cell injury. Specific NSAIDS approved for administration to humans include naproxen sodium, diclofenac, sulindac, oxaprozin, diflunisal, aspirin, piroxicam, indomethacin, etodolac, ibuprofen, fenoprofen, ketoprofen, mefenamic acid, nabumetone, tolmetin sodium, and ketorolac tromethamine. Anti-Inflammatory agents are a well-known class of pharmaceutical agents which reduce inflammation by acting on body mechanisms (Stedman's Medical Dictionary 26 ed., Williams and Wilkins, (1995); Physicians' Desk Reference 51 ed., Medical Economics, (1997)).

In some embodiments, the mobilization fluid may further contain additional agents, such as preservatives, viscosity adjusting additives, and other potentially beneficial materials, such hydrogen peroxide or hemoglobin derived oxygen carriers. Any volume of the formulated mobilization fluid may be delivered as needed to the treated dermis in order to effectively facilitate removal of the dislodged and degraded ink particles and by-products thereof during the extraction step. In preferred embodiments the total volume of mobilization fluid used to remove dislodged and degraded ink particles and degradation by-products thereof is less than about 10 mL, more preferably less that about 5 mL, even more preferably less than about 2 mL, and most preferably less than about 1 mL.

In one non-limiting embodiment a tattoo removal system can include liquid source, a gas source and a mixer. The system can optionally include a pump and/or a temperature regulator. The system can be operated under the control of a controller. The system can operate, in one example, to deliver a liquid-gas mixture to the target site. To generate a plasma within the gas component, a power source (also under control of the controller) can deliver a DC and/or AC current to an electrical conductor of a handpiece adaptor. In certain embodiments, the electrode can be a dielectric barrier discharge electrode. In other embodiments, the discharge can be induced by a piezoelectric transducer.

The system can further include an optional vacuum source to extract dislodged and/or degraded tattoo ink particles. (The vacuum or suction source can also be a stand-alone instrument in some embodiments of the system).

The system can further include an alternative liquid supply. This alternative liquid supply can provide an additional source of mobilization fluid that can be provided to the target tattoo region before, during or after plasma treatment.

The gas or liquid/gas mixture can then be delivered to one or more hollow needles configured to penetrate the epidermis and deliver the plasma to the dermis where it can act upon tattoo ink particles and/or cellular structures in which the ink particles are embedded, encased or otherwise bound. The cold plasma can be formed by corona discharge or dielectric barrier discharge.

An apparatus for generating cold plasma via excited gas bubbles can comprise the above-described adaptor and one or more needles. A source of liquid and a source of gas can be fed into a mixing unit arranged upstream of the fluid supply line to deliver the liquid with entrained gas to the handpiece adaptor. The adaptor can be coupled to a hollow needle or array of needles to deliver the liquid with cold plasma gas bubbles to a target site. The adaptor unit and the needle or needle array can be separate components or integrally formed together.

In certain embodiments, the adaptor can be coupled to a plasma or non-plasma source for generating a high energy alternating electric field at the distal end of the adaptor. A controller can determine the proportions of the at least one gaseous substance and/or of the at least one liquid/gas mixture substance or of the various liquid substances, so that a desired mixture enters the plasma discharge adaptor. The controller can also adjust the voltage applied to the electrical conductor of the adaptor.

The applied alternating electric field is preferably strong enough that a cold gas discharge can be ignited in the gas or inside gas bubbles in a liquid. The intensity of the interaction can be controlled by the amplitude and frequency of the applied alternating electric field, the mixing ratio and the total surface area of the interfaces between the gaseous and liquid phases. Many liquids have a dielectric constant significantly greater than 2. Typical gases have a dielectric constant of nearly 1. Water, for example, has a dielectric constant of 80. This difference in dielectric constant between a gaseous substance and a liquid substance can cause a sharp drop in the applied electrical potential in the gas bubble. As a result, the electric field strength in the gas bubbles is particularly high.

In certain embodiments, the mechanism for generating the alternating electric field within the tubular discharge unit can be a piezoelectric transducer or transformer. In other embodiments the mechanism can be a dielectric barrier electrode. In either embodiment, at least one electrode is coupled to the plasma discharge unit. The electrode can be connected to a voltage source and the voltage applied is controlled by a controller. An electric field can be formed at the needle tip or can extend beyond the needle tip.

The systems disclosed herein can be configured in such a way that a spray at the at the end of a needle or needle array is dispersed or atomized by electrostatic charging. In certain embodiments, the spray is delivered to a tattooed region of the dermis.

A mixing unit may be connected to at least one source or reservoir for the at least one gaseous or liquid substance. A plurality of reservoirs for the liquid and/or gaseous substance can be connected to the mixing unit.

A temperature control device (a heater or cooling unit) can also be included in the system whereby the active species in the fluid supply line can be heated and/or cooled. With the temperature control device, the temperature of the gas or a mixture of the at least one gaseous substance and the at least one liquid substance at the target site can thus be brought to a predetermined value. In tattoo removal applications, the discharge can be controlled such that the plasma is maintained at or near room temperature (e.g., a cold plasma).

When a liquid/gas mixture is used, the mixture can be pumped from a mixing unit into the fluid supply line. In the energy conductor or at the needle tip(s), bubbles of the gaseous substance can be exposed to an intense electric field. In certain embodiments, a controller generates an alternating electric field which is applied to the mixture of the at least one gaseous substance and of the at least one liquid substance. The gas discharge is ignited in the bubbles formed by the gaseous substance. The mixing ratio of the at least one gaseous substance and the at least one liquid substance can be adjusted or set by the controller. A controller can also set the applied strength of the alternating electric field. The mixing ratio and the total surface area of the interfaces between the gaseous and liquid phases can be adjusted by means of the intensity of the interaction, the amplitude and the frequency of the applied alternating electric field. The active species generated by the alternating electric field are fed to a target tattoo region via a second end of the discharge unit.

The intensity of electric field generated at the needle tip can be controlled by adjusting a DC voltage source. The gaseous and/or liquid/gas mixture can be forced through the needle or needle array in such a way that a stream or dispersed spray is delivered to a target dermal region where tattoo ink particles are located. The flow of liquid with entrained plasma gas bubbles from the needles can be continuous or discontinuous.

The inventive methods can be employed to stimulate the skin and/or subcutaneous areas with an alternating electric field and the cold plasma. It is also possible to flush a subcutaneous cavity with an activated liquid or mixture of different liquids (liquid substances) or an activated gas mixture or a mixture of at least one gaseous substance and at least one liquid substance. Furthermore, an electrophoretic effect may occur which promotes the mobility of particles in the dermis. The method according to aspects of the disclosure can also be used to degrade, dislodge, rinse, wash out or otherwise render tattoo ink particles in a target dermal region less discernible.

The method according to aspects of the disclosure allows the chemical properties of tattoo ink particles to be changed when they come into contact with the cold plasma. For example, biological structures in the skin or elsewhere, such as various cells, collagens, or other structural proteins, can be influenced and treated. Non-biological material in the skin, such as tattoos or other foreign bodies, can be degraded and/or dislodged.

The gaseous substance used in the method according to aspects of the disclosure may be, for example, ambient air, a noble gas, oxygen, nitrogen, carbon dioxide or a mixture of these gases. The liquid substance can be water, a salt solution, an alcohol, H2O2 or a mixture of the above liquids. Additives such as antibiotics, reactive monomers, surfactants or foaming agents can also be added to the liquid substance.

In certain embodiments, discrete bubbles of the gaseous substance form in the liquid substance in the energy conductor or at the needle tip. Here the ratio between the proportion of the gaseous substance and the proportion of the liquid substance is controlled in such a way that individual discrete bubbles form within the liquid and are activated as a plasma or highly active gas species. The temperature of this mixture of the liquid substance and the activated gaseous substance can be adjusted to a desired value by heating or cooling.

At least one component, the liquid or the gaseous component, can be collected after passing through the discharge zone and can be passed on to further use. The apparatus according to aspects of the disclosure can be adapted or controlled to change the density and volume of the gaseous and/or liquid substances in the tubular discharge unit (e.g., a needle or probe-like structure coupled to a plasma generating handpiece).

In certain embodiments, a discharge system can be used for heterogeneous mixtures of at least one gas and at least one liquid, or also of special liquids without the admixture of a gas, to produce active species in the gas or liquid phase. The active species can be used in various applications such as liquid sterilization, surface treatment, coating, human or animal skin treatment such as direct subepidermal CAP treatment, and many others.

In another non-limiting embodiment, a system for tattoo removal includes a main housing wherein: a plasma generation component; a fluid delivery component; and a fluid extraction component are integrated. In some other embodiments, the fluid delivery component may be excluded from the system. The system is connected and coupled to a free-standing plasma generating handpiece, which may be in the form of pen or wand-like component. The housing of the tattoo removal system also includes additional components, as needed, to power the aforementioned components and the handpiece, so as to provide power from an electrical outlet or from one or more battery source(s). The main housing may further include one or more control unit(s), which may include input controls (i.e., knobs, buttons, foot pedals) and analog or digital displays which show parameters of the components in order to control and regulate each component's parameters prior to and during operation. In some embodiments, one (main) control unit may be used to control all the components, while in some other embodiments each component has its own individual control unit on the system's main housing.

In some other embodiments, the plasma generation component; a fluid delivery component; and a fluid extraction component can be incorporated into a single combined treatment system. In some embodiments, the fluid delivery component may be excluded from the combined treatment component. A foot pedal can provide means for controlling the plasma application, saline wash, and extraction.

In yet other embodiments, a kinetic applicator can be incorporated into the treatment components (handpiece or adaptor) of aspects of the disclosure, including a motor and cam mechanism to impart a vibratory or oscillating motion to the needle.

The electrical power source component may be a commercially available component which is adapted to be a part of the tattoo removal system described herein. The plasma generation component housed in the main system and/or handpiece can include all necessary components required to provide a high frequency alternating current, or high repetition rate pulsed direct current to one or more skin-penetrating needles. Optional components relating to cold plasma formation can also include, but are not limited to, gas inputs, valves, regulators, pumps, gas mixing chamber/units, power systems. The conditions, such as the power, flow rate of gas(es), and the ratio of gases in mixtures used to generate a cold plasma can be controlled as needed to achieve the desired properties of the cold plasma, using the input control(s) connected and coupled to the plasma generation unit.

Typically, plasmas are generated from a gas or a mixture of gases which may include, but are not limited to, air, carbon dioxide, oxygen, nitrogen, helium, argon, neon, xenon, and krypton. In preferred embodiments, the cold plasma generation unit receives gas(es) from one or more gas sources. In some embodiments, the one or more gas sources may be in the form of free-standing replaceable gas tanks/cylinders or the one or more gas(es) may be from a source such as a gas outlet present on a wall and connected to a central gas source. In certain embodiments, the one or more gas sources are external to the main housing of the tattoo removal system and are coupled and connected to the one or more gas inputs of the plasma generation component of the system by any suitable means (i.e., gas regulator and gas tubing). In certain other embodiments, the one or more gas sources may be included within the housing of the tattoo removal system, if desirable. In preferred embodiments the power used to generate the cold plasma is in the range of about 80 W to about 150 W. In some preferred embodiments, the gas flow rates are in the range of about 0.00001 to about 15 L min-1. The relative percentages of the one or more gases present in the mixture can be controlled by a gas mixing unit to achieve any suitable relative gas mix percentage necessary to achieve a cold plasma. In certain embodiments, wherein the plasma generating mixture of gases is composed of oxygen mixed with argon or helium, the percentage of oxygen in the mixture is preferably in the range of about 0.1% to about 5%.

The plasma generation component is coupled and connected using any suitable means and outputs/delivers the cold plasma generated to the treatment component for delivery to the tattooed dermis. The cold plasma stream generated may be controlled via the one or more input control units of the system. The plasma output by the plasma generation component to the treatment component may be a continuous cold plasma jet stream or a discontinuous pulsed cold plasma jet stream. It should be apparent that the details described herein are non-limiting and that other suitable conditions and parameters can be selected and utilized in order to generate and deliver the cold plasma to the tattooed dermis. The delivery of cold plasma to the dermis via a treatment component, which may be in the form of a pen/wand, can be controlled by a skilled/trained operator or technician using an input control unit, such as a foot pedal.

The fluid delivery component of the system includes one or more fluid reservoir units which can hold one or more liquids to be mixed with the ionized gas or to serve a separate mobilization fluid. The one or more reservoir units are coupled and connected to the treatment component of the tattoo removal system by any suitable means (i.e., tubing) in order to output the mobilization fluid to the treatment component. The mobilization fluid delivery component includes one or more controllable fluid pumps which deliver the mobilization fluid to the treatment component at a controllable flow rate. The flow rate of the fluid can be regulated by the one or more input controls or units coupled and connected to the fluid delivery component. In some embodiments the mobilization fluid is not pre-formulated but can be generated on-demand by mixing units which may form part of the fluid delivery component. Such mixing units are fed by the one or more fluid reservoir units which may contain the component fluids and other agents which form the desired mobilization fluid such as, but not limited to, sterile water, saline solution, buffered aqueous solutions and suitable local anesthetics, anti-infective agents, antiseptic agents, anti-inflammatory agents, and combinations thereof. The delivery of mobilization fluid to the dermis via the treatment component can be controlled by a skilled/trained operator or technician using an input control unit, such as a foot pedal.

In some other embodiments, the fluid delivery component, as described above, may be directly incorporated into a free-standing pen or wand-like component. In such embodiments, one or more disposable fluid cartridges which hold a given volume of pre-formulated mobilization fluid (described above) may be coupled and connected to the fluid delivery component to output the mobilization fluid to one or more needle or probe-like structures of the treatment component as described below. In such embodiments, the delivery of mobilization fluid to the dermis via the one or more needle or probe-like structures of the treatment component can be controlled by a skilled/trained operator or technician using an input control unit present on the treatment component.

The fluid extraction component of the system includes one or more vacuum pumps and/or other components necessary for creating a vacuum or partial vacuum and is connected and coupled by any suitable means to the treatment component so as to create suction used to extract the mobilization fluid delivered to the dermis during tattoo removal treatment and draw/extract the mobilization fluid containing dislodged and degraded ink particles and by-products thereof, and tissue by-products thereof away from the dermis and surrounding tissues of the subject. In some embodiments of the system which exclude a fluid delivery component and mobilization fluid, the fluid extraction component can remove the dislodged degraded tattoo ink particles which may be present in the natural fluids of the dermis or surrounding tissue directly. In some embodiments, suction created by the extraction component is applied as a continuous suction or, alternatively, the suction can be applied intermittently. The application of suction to the dermis and/or surrounding tissue can be controlled by a skilled/trained operator or technician using an input control unit, such as a foot pedal.

In some other embodiments, the fluid extraction component, as described above, may be directly incorporated into the plasma generating handpiece or the needle adaptor. In such embodiments, the application of suction to the dermis and/or surrounding tissue can be controlled by a skilled/trained operator or technician using an input control unit present on the handpiece.

The treatment component (e.g., the handpiece and adaptor) can be coupled and connected to the other components discussed above using any suitable means known. The treatment component can include suitable mechanical components, as needed, to deliver electrical energy (and, optionally, cold plasma) and mobilization fluid into the dermis and to apply suction to the dermis. One end of the treatment component may include one or more inputs and outputs (not shown) which are connected/coupled to the other components of the system as described above when these components are external to the treatment component. For example, the inputs can receive the electrical energy and mobilization fluid and the output can receive the mobilization or other body fluid extracted from the dermis or surrounding tissue during tattoo removal. The opposite end of the treatment component includes a treatment end which can output and deliver the activated liquid-gas mixture and/or mobilization fluid into the dermis. The treatment end can also receive extracted mobilization fluid, or other natural body fluids, which contain dislodged and degraded tattoo ink particles during treatment of the dermis and surrounding tissue.

In certain embodiments, the treatment component also includes a cartridge unit which contains one or more needle or probe-like structures, which penetrate the subject's tattooed skin, such that the needle(s) form part of a removable, disposable, and/or replaceable unit cartridge. The one or more needle or probe-like structures can be made of either plastic, metal or a combination thereof. In some non-limiting embodiments, the removable, disposable, and/or replaceable cartridge includes one, two, three, four, five, six, seven or more needles. The depth of penetration of the one or more needle or probe-like structures, present in the needle cartridge, into the skin is preferably to the depth of the dermis of the subject's tattooed skin but may be adjusted by a skilled/trained operator or technician as needed to apply the tattoo removal treatment method using the system described herein. The one or more needle or probe-like structures, which penetrate into the tattooed dermis, can oscillate or pulse during tattoo removal treatment via a mechanical process, such as a piston like drive which pulses and/or oscillates the needles in and out of the dermis at varying speeds. In certain other embodiments, the one or more needle or probe-like structures, which penetrate into the tattooed dermis are fixed and do not pulse or oscillate.

In some embodiments, the one or more needle or probe-like structures oscillate or pulse and with each oscillation or pulse perform one or more functions of delivering electrical energy, delivering cold plasma, delivering mobilization fluid to the dermis, or extracting the mobilization fluid containing dislodged and degraded ink particles and by-products thereof, and tissue by-products thereof. In some embodiments each full or partial oscillation or pulse applies a particular function sequentially at a time and all the functions as described are performed so as to provide at least one complete cycle which includes the dislodgement, mobilization, and extraction steps. In certain other embodiments, all of the functions are applied concurrently during a given oscillation or pulse of the one or more needles. In some other embodiments, some, but not necessarily all of, the functions described form part of a cycle which is performed at least one or more times during a given oscillation or pulse of the one or more needles, as necessary to remove the tattoo ink from the subject's dermis and rendering the tattoo undetectable, invisible, and/or non-discernible. Each of the needle or probe-like structures of the removable, disposable, and/or replaceable unit cartridge can be formed of a multiple sheathed needle which is formed from nested multiple concentric needles.

In certain embodiments, a multi-sheathed needle or probe-like is formed of three concentric nested/embedded needle or probe-like structures forming inner, middle, and outer tubes. In some embodiments, the outer most tube can deliver the active species for cold plasma ignition and optionally the outer most portion of the needle or probe-like structure includes suitable openings 308 on the outer side for delivering cold plasma to the dermis. In some embodiments, the middle tube delivers mobilization fluid to the dermis. In some embodiments, the inner most tube provides suction to the dermis to remove mobilization fluid containing dislodged and degraded tattoo ink particles and by-products thereof from the dermis. Any one or all of the concentric structures can serve as the active electrode for delivery of electrical energy.

In another non-limiting example, a multi-sheathed needle can be formed of two concentric nested/embedded needle or probe-like structures forming inner and outer tubes. In some embodiments, the outer most tube delivers cold plasma and extraction fluid which are sequentially pulsed into the dermis. Optionally, the outer most portion of the needle or probe-like structure can include suitable openings on the outer side for delivering cold plasma to more than one region of the dermis. In some embodiments, the inner tube provides suction to the dermis to remove mobilization fluid containing dislodged and degraded tattoo ink particles and by-products thereof from the dermis. Again, anyone or both of the concentric structures can serve as the active electrode for delivery of electrical energy.

In another non-limiting example, a single-sheathed needle may be used. Optionally, the outer surface of the needle or probe-like structure can include suitable openings on the outer side for delivering cold plasma to multiple regions of the dermis. In a single sheath configuration, the cold plasma, mobilization fluid, and suction are sequentially applied to the dermis during treatment and the sheath itself is conductive for delivery of electrical energy to the target tattoo region.

One of ordinary skill will immediately recognize that the above examples are non-limiting and variations are permitted regarding the use of any of the sheaths present in the embedded/nested structure to achieve any of the plasma, fluid, or extraction functions as described above. In some embodiments, the rate of flow of cold plasma, mobilization fluid and rate of suction can be controlled by a computerized flow meter included in the treatment component.

In some embodiments, an input control, such as a foot pedal or button(s) present on the treatment component, may be used to activate, deactivate, and control all of electrical energy, cold plasma, dislodgement, mobilization and extraction components coupled and connected to the treatment component, or integrated within the treatment component which may be in the form of a pen/wand, at one time or may control the electrical energy, cold plasma, dislodgement, mobilization and extraction components individually. In some other embodiments, an input control, such as a foot pedal and/or button(s) present on the treatment component, can be used initiate a cycle which triggers each function of a given component in a given sequence. The cycle/sequence may be repeated at any suitable interval of time and for any suitable number of cycles as needed to remove the tattoo from the subject's dermis and surrounding tissue.

The application of electrical energy, plasma, mobilization fluid, and/or extraction (i.e., suction) through the one or more needle can be done manually or via a predetermined program. In some embodiments, the operator/technician may apply electrical energy and depending on the extent to which the tattoo has been rendered undetectable, invisible, and/or non-discernible determine not to apply cold plasma, a mobilization fluid and actuate extraction. In certain other embodiments, the operator/technician may choose to further apply a mobilization fluid and extraction in order to further render the tattoo undetectable, invisible, and/or non-discernible. In yet another embodiment, the operator/technician may choose to only further apply extraction to remove dislodged and degraded tattoo ink particles, degradation by-products thereof, and/or tissue by-products thereof contained in bodily fluid without applying a mobilization fluid.

The adaptor and methods disclosed herein have been described in detail in connection with one exemplary use, i.e., tattoo removal. However, the adaptors can also be used for other purposes whenever it is desirable to deliver a plasma to a subsurface or hard-to-reach site.

Unless defined otherwise, all technical and scientific terms used herein have the same meanings as commonly understood by one of skill in the art to which the present teachings belong. All patents, patent applications and publications of any kind cited herein and the materials for which they are cited are specifically incorporated by reference in their entirety.

It should be understood that any method step or element described herein can be used in conjunction with any other method or element, respectively, whether or not such combination is described in a specific example or embodiment. All such permutations are embraced as part and parcel of aspects of the disclosure.

Within this specification, embodiments have been described in a way that enables a clear and concise specification to be written, but it is intended and will be appreciated that embodiments may be variously combined or separated without departing from the scope of the disclosure. For example, it will be appreciated that all preferred features (element or method step) described and/or claimed are applicable to all aspects of the disclosure described herein. Every claimed feature should be deemed capable of multiple dependencies from other claimed features even if only one dependency is recited unless the combination of features is physically impossible.

Those skilled in the art will recognize, or be able to ascertain using no more than routine experimentation, many equivalents to the specific embodiments of the exemplary teachings described herein. Such equivalents are also intended to be encompassed by the following claims.

### ELEMENTS OF THE DRAWINGS

2. Plasma Generating Handpiece
3. Handpiece Body
4. Piezoelectric Crystal
6. Handpiece controls
7. Power Supply Line
8. Power Source
9. Adaptor Release Clip
10. Adaptor
12. Fluid Supply Line
13. Fluid Supply Line Lumen
14. Needle
15. Needle Lumen
16. Needle Collar
18. Air Inlet Ports
20. Energy Conductor (“Tack”)
22. Fluid Line Connector
24. Proximal Adaptor-to-Handpiece (Slip-Lock) Coupler
26. Distal (Male) Threaded Needle Connector
28. Adaptor Body
30. Conductor Plate (“Tack Head”)
32. Side (Female) Threaded Fluid Line Coupler
34. Lateral Lumen (through Energy Conductor)
36. Longitudinal Lumen through Energy Conductor)
38. Initial Plasma at the Tack Head
40. Plasma at the Needle Tip

We claim:

1. A needle adaptor for connection to a plasma generating handpiece, the adaptor comprising:
an adaptor body having a proximal handpiece coupler for coupling to a plasma generating handpiece;
a needle connector at a distal end of the adaptor body for connection to one or more needles;
an energy conductor disposed at least partially within the adaptor body and extending longitudinally between the plasma generating handpiece and the needle connector; and
a fluid line connector for connection to a fluid supply line;
wherein the energy conductor further comprises at least one internal fluid passageway such that a fluid can flow through the energy conductor to the needle coupler and the fluid and plasma generating energy can be directed via at least one needle to a target site.

2. The needle adaptor of claim 1, wherein the handpiece coupler of the adaptor body further comprises at least one annular groove, annular lip, threaded connector or luer-lock type connector for coupling to a mating connector on the handpiece.

3. The needle adaptor of claim 1, wherein the needle connector further comprises a threaded connection that couples to a mating connection on a needle.

4. The needle adaptor of claim 1, wherein the needle connector comprises a male threaded segment of the energy conductor that couples to a female threaded collar that is joined to one or more needles.

5. The needle adaptor of claim 1, wherein the adaptor body further comprises a threaded connection that couples to a mating connector on the fluid supply line.

6. The needle adaptor of claim 5, wherein the adaptor body further comprises a female threaded connection that couples to a mating male threaded connector on the fluid supply line, or vice-versa.

7. The needle adaptor of claim 1, wherein the energy conductor further comprises a longitudinal lumen configured to deliver fluid to at least one needle.

8. The needle adaptor of claim 1, wherein the energy conductor further comprises at least one lateral lumen to receive fluid from the fluid delivery line and deliver the fluid to the longitudinal lumen of the energy conductor.

9. The needle adaptor of claim 1, wherein the adaptor further comprises at least one needle having a collar configured to connect to the needle connector of the adaptor body.

10. A needle adaptor for connection to a plasma generating handpiece, the adaptor comprising:
an adaptor body having a proximal handpiece coupler for coupling to a plasma generating handpiece and a threaded connection that couples to a mating connector on a fluid supply line;
a needle connector at a distal end of the adaptor body for connection to one or more needles, wherein the needle connector further comprises a threaded connection that couples to a mating connection on a needle;
an energy conductor disposed at least partially within the adaptor body and extending longitudinally between the plasma generating handpiece and the needle coupler; and
a fluid line connector for connection to a fluid supply line;
wherein the energy conductor further comprises at least one internal fluid passageway such that a fluid can flow through the energy conductor to the needle coupler and the fluid and plasma generating energy can be directed via at least one needle to a target site.

11. A method of delivering plasma and at least one fluid to a target site, the method comprising:
connecting a needle adaptor to plasma generating handpiece, the needle adaptor comprising an adaptor body having a proximal handpiece coupler for coupling to a plasma generating handpiece, a needle connector at a distal end of the adaptor body for connection to at least one needle, an energy conductor disposed at least partially within the adaptor body and extending longitudinally between the plasma generating handpiece and the needle coupler; and a fluid line connector for connection to a fluid supply line;
connecting a fluid supply line to the fluid line connector of the adaptor;
disposing a distal end of said at least one needle at a target site;
delivering fluid from the fluid supply line to a target site via an internal fluid passageway in the energy conductor; and
activating the plasma generating handpiece to deliver energy to a said at least one needle connected to the distal end of the energy conductor,
whereby a plasma is formed at the distal end of the needle.

12. The method of claim 11, wherein the fluid comprises a gas.

13. The method of claim 12, wherein the gas comprises at least one gas selected from air, carbon dioxide, oxygen, nitrogen, helium, argon, neon, xenon, and krypton.

14. The method of claim 11, wherein the fluid comprises a liquid.

15. The method of claim 14, wherein the liquid comprises at least one liquid selected from water, saline, and buffered aqueous solutions.

16. The method of claim 11, wherein the fluid comprises a liquid-gas mixture.

17. The method of claim 11, wherein the plasma comprises a cold atmospheric plasma.

18. The method of claim 11, wherein the target site is a tattooed region of a subject's dermis.

19. The method of claim 18, wherein the method further comprises pretreating the target region by injecting the region with a mobilization fluid via the needle adaptor before plasma generation.

20. The method of claim 18, wherein the method further comprises applying suction to the target region to extract degraded or dislodged tattoo ink particles following plasma generation.
